(19) United States
(12) Patent Application Publication
(10) Pub. No.: US 2020/0169724 A1
(43) Pub. Date: May 28, 2020

(54) OPTIMIZING PERCEPTION OF STEREOSCOPIC VISUAL CONTENT

(71) Applicant: NUTRICERN, INC., Ssbastopol, CA (US)

(72) Inventor: Dwight Meglan, Westwood, MA (US)

(21) Appl. No.: 16/634,284

(22) PCT Filed: Aug. 16, 2018
(86) PCT No.: PCT/US2018/000290

§ 371 Date: Jan. 27, 2020

Related U.S. Application Data
(60) Provisional application No. 62/546,306, filed on Aug. 16, 2017.

(51) Int. Cl. H04N 13/337; G02B 27/01; A61B 34/30

(52) U.S. Cl. CPC H04N 13/337; G02B 27/0172; G02B 2027/0178; G02B 2027/0134; A61B 34/30

(57) ABSTRACT

Systems, methods, and computer-readable media are provided for controlling a stereoscopic display device to improve perception of stereoscopic visual content. One method includes displaying a first item of visual content through a first portion of a polarizing filter, and displaying, through the first portion and/or a second portion of the polarizing filter, a first message based on whether the first item of visual content is to be visible by a first or second eye of the observer. Another method includes capturing an image of an observer, determining a position of the observer based on the captured image, comparing the determined position to a predetermined position criterion, and causing a message based on a result of the comparing to be provided to the observer. In another method, the display device is repositioned based on a result of the comparing.

### BACKGROUND

Stereoscopic displays, sometimes referred to as three-dimensional (3D) displays, are employed in numerous settings to enable observers to perceive depth in presented images. For instance, a stereoscopic display may be used by a clinician as part of a robotic surgical system. A stereoscopic display facilitates depth perception in an image by presenting the image to the observer as a pair of distinct images separately provided to the left and right eyes, respectively. The pairs of images are created to replicate the effect of the offset between the left and right eyes, which results in a difference in what is seen in the display by each eye. The different images seen in the display by each eye are perceived as differences in the depths of the objects in the images, for example, as a result of the varying of image offsets in different areas of the display based on the depth of the object to be observed.

A typical passive stereoscopic display includes a film carefully aligned with the pixels of the display that, in conjunction with a corresponding pair of stereoscopic eyeglasses worn by the observer, enables certain pixel rows to be visible by one eye and other pixel rows to be visible by the other eye. In particular, the film filters certain pixels of the display (in an example, the odd pixel rows) according to a first type of polarization and filters other pixels of the display (in an example, the even pixel rows) according to a second type of polarization. The left lens of the eyeglasses is matched to the first type of polarization and is designed to permit visual content polarized according to the first type of polarization to reach the left eye and prevent visual content polarized according to the second type of polarization from reaching the left eye. The right lens of the eyeglasses is matched to the second type of polarization and is designed to permit visual content polarized according to the second type of polarization to reach the right eye and prevent visual content polarized according to the first type of polarization from reaching the right eye. In this way, the display can provide a first image to one of the eyes by way of the odd pixel rows, and provide a second image to the other eye by way of the even pixel rows.

The stereoscopic display scheme described above can work well when the eyes of the observer are positioned in a proper position and orientation, within certain tolerance amounts, relative to the plane of the display. However, when the eyes of the observer are positioned beyond the tolerance amounts, for example in an improper position and/or orientation relative to the ideal position with respect to the plane of the display, the observer's perception of the visual content is degraded because portions of the images intended for a particular eye reach, and are perceived by, the other eye and vice versa. This misalignment causes the observer to experience a phenomenon known as ghosting. As such, there is a need for systems and methods that improve perception of stereoscopic visual content by optimizing the positional relationship between the eyes of the observer and the stereoscopic display.

### SUMMARY

According to an aspect of the present disclosure, a stereoscopic display system is provided that includes a display device, a polarizing filter, a memory storing instructions, and a processor configured to execute the instructions. The display device includes a first set of pixels and a second set of pixels. The polarizing filter is affixed to, or integrated with, the display device, and includes a first portion that filters visual content according to a first polarization and is aligned with the first plurality of pixels of the display device, and a second portion that filters visual content according to a second polarization and is aligned with the second plurality of pixels of the display device. The processor executes the instructions to cause the display device to display a first item of visual content through the first portion of the polarizing filter, and display a first message through the first and/or second portion of the polarizing filter. The first message is based at least in part on whether the first item of visual content is intended to be visible by a first eye of an observer or a second eye of the observer.

In another aspect of the present disclosure, the processor is further configured to execute the instructions to cause the display device to display a second item of visual content through the second portion of the polarizing filter, and display a second message through the first and/or second portion of the polarizing filter. The second message is based on whether the second item of visual content is intended to be visible by the first eye of the observer or the second eye of the observer.

In still another aspect of the present disclosure, one or more of the first item of visual content, the second item of visual content, the first message, and/or the second message is concurrently displayed with another one or more of the first item of visual content, the second item of visual content, the first message, and/or the second message.

In another aspect of the present disclosure, one or more of the first item of visual content and/or the second item of visual content includes one or more of a predetermined color, a predetermined pattern, and/or predetermined textual content, and the first item of visual content is distinct from the second item of visual content.

In another aspect of the present disclosure, the first message includes one or more of a query and/or an instruction relating to repositioning of the eyes of the observer so that the first item of visual content is visible by the first eye of the observer but not visible by the second eye of the observer. The second message includes one or more of a query and/or an instruction relating to repositioning of the eyes of the observer so that the second item of visual content is visible by the second eye of the observer but not visible by the first eye of the observer.

In another aspect of the present disclosure, the first polarization is clockwise circular polarization and the second polarization is counterclockwise circular polarization, or the first polarization is a first linear polarization and the second polarization is a second linear polarization ninety degrees out of phase with respect to the first linear polarization.

According to another aspect of the present disclosure, a computer-implemented method for controlling a stereoscopic display device is provided. The method includes displaying a first item of visual content through a first portion of a polarizing filter, and displaying, through the first and/or second portion of the polarizing filter a first message. The first portion of the polarizing filter filters visual content according to a first polarization and is aligned with a first plurality of pixels of a display device, and the second portion of the polarizing filter filters visual content according to a second polarization and is aligned with a second plurality of pixels of the display device. The first message is based on whether the first item of visual content is intended to be visible by a first eye of an observer or a second eye of the observer.

In another aspect of the present disclosure, the computer-implemented method further includes displaying a second item of visual content through the second portion of the polarizing filter, and displaying a second message through the first and/or second portion of the polarizing filter. The second message is based on whether the second item of visual content is intended to be visible by the first eye of the observer or the second eye of the observer.

In still another aspect of the present disclosure, one or more of the first item of visual content, the second item of visual content, the first message, and/or the second message is concurrently displayed with another one or more of the first item of visual content, the second item of visual content, the first message, and/or the second message.

In another aspect of the present disclosure, one or more of the first item of visual content and/or the second item of visual content includes one or more of a predetermined color, a predetermined pattern, and/or predetermined textual content, and the first item of visual content is distinct from the second item of visual content.

In another aspect of the present disclosure, the first message includes one or more of a query and/or an instruction relating to repositioning of the eyes of the observer so that the first item of visual content is visible by the first eye of the observer but not visible by the second eye of the observer. The second message includes one or more of a query and/or an instruction relating to repositioning of the eyes of the observer so that the second item of visual content is visible by the second eye of the observer but not visible by the first eye of the observer.

In another aspect of the present disclosure, the first polarization is clockwise circular polarization and the second polarization is counterclockwise circular polarization, or the first polarization is a first linear polarization and the second polarization is a second linear polarization ninety degrees out of phase with respect to the first linear polarization.

According to another aspect of the present disclosure, a non-transitory computer-readable medium is provided. The non-transitory computer-readable medium stores thereon instructions which, when executed by a processor, cause a display device to display a first item of visual content through a first portion of a polarizing filter, and display a first message through the first portion of the polarizing filter and/or through a second portion of the polarizing filter. The first message is based on whether the first item of visual content is intended to be visible by a first eye of an observer or a second eye of the observer. The first portion of the polarizing filter filters visual content according to a first polarization and is aligned with a first plurality of pixels of a display device, and the second portion of the polarizing filter filters visual content according to a second polarization and is aligned with a second plurality of pixels of the display device.

In another aspect of the present disclosure, the instructions, when executed by the processor, further cause the display device to display a second item of visual content through the second portion of the polarizing filter, and display a second message through the first and/or second portion of the polarizing filter. The second message is based on whether the second item of visual content is intended to be visible by the first eye of the observer or the second eye of the observer.

In still another aspect of the present disclosure, one or more of the first item of visual content, the second item of visual content, the first message, and/or the second message is concurrently displayed with another one or more of the first item of visual content, the second item of visual content, the first message, and/or the second message.

In another aspect of the present disclosure, one or more of the first item of visual content and/or the second item of visual content includes one or more of a predetermined color, a predetermined pattern, and/or predetermined textual content, and the first item of visual content is distinct from the second item of visual content.

In another aspect of the present disclosure, the first message includes one or more of a query and/or an instruction relating to repositioning of the eyes of the observer so that the first item of visual content is visible by the first eye of the observer but not visible by the second eye of the observer. The second message includes one or more of a query and/or an instruction relating to repositioning of the eyes of the observer so that the second item of visual content is visible by the second eye of the observer but not visible by the first eye of the observer.

In another aspect of the present disclosure, the first polarization is clockwise circular polarization and the second polarization is counterclockwise circular polarization, or the first polarization is a first linear polarization and the second polarization is a second linear polarization ninety degrees out of phase with respect to the first linear polarization.

According to another aspect of the present disclosure, a system for improving perception of stereoscopic visual content is provided that includes an image capture device configured to capture an image of an observer, and a processor configured to determine a position of the observer based on the captured image, compare the determined position of the observer to a predetermined position criterion, and cause a message based on a result of the comparing to be provided to the observer.

In another aspect of the present disclosure, the system further includes one or more of a display device and/or an audio device. The display device is configured to, in a case where the result of the comparing indicates that the position of the observer should be corrected for improved perception of stereoscopic visual content, provide the message in the form of display content indicating a direction in which the observer should move to correct position for improved perception of stereoscopic visual content. The audio device is configured to, in a case where the result of the comparing indicates that the position of the observer should be corrected for improved perception of stereoscopic visual content, provide the message in the form of audio content indicating a direction in which the observer should move to correct position for improved perception of stereoscopic visual content.

In still another aspect of the present disclosure, the determining the position of the observer includes determining a relative position of one or more of an eye of the observer, stereoscopic eyeglasses worn by the observer, and/or a head of the observer, with respect to one or more of the image capture device and/or the display device.

In another aspect of the present disclosure, the system further includes one or more of a display device and an audio device. The display device is configured to, in a case where the result of the comparing indicates that the observer is correctly positioned for perception of stereoscopic visual content, provide the message in the form of display content indicating that the observer is correctly positioned for perception of stereoscopic visual content. The audio device is configured to, in a case where the result of the comparing indicates that the observer is correctly positioned for perception of stereoscopic visual content, provide the message in the form of audio content indicating that the observer is correctly positioned for perception of stereoscopic visual content.

In another aspect of the present disclosure, the predetermined position criterion includes a range of acceptable observer positions for perception of stereoscopic visual content or a range of unacceptable observer positions for perception of stereoscopic visual content.

According to another aspect of the present disclosure, a computer-implemented method for improving perception of stereoscopic visual content is provided. The method includes capturing an image of an observer, determining a position of the observer based on the captured image, comparing the determined position of the observer to a predetermined position criterion, and causing a message based on a result of the comparing to be provided to the observer.

In another aspect of the present disclosure, the computer-implemented method further includes one or more of: (1) in a case where the result of the comparing indicates that the position of the observer should be corrected for improved perception of stereoscopic visual content, providing the message in the form of display content indicating a direction in which the observer should move to correct position for improved perception of stereoscopic visual content; and/or (2) in a case where the result of the comparing indicates that the position of the observer should be corrected for improved perception of stereoscopic visual content, providing the message in the form of audio content indicating a direction in which the observer should move to correct position for improved perception of stereoscopic visual content.

In still another aspect of the present disclosure, the determining the position of the observer includes determining a relative position of one or more of an eye of the observer, stereoscopic eyeglasses worn by the observer, and/or a head of the observer, with respect to one or more of an image capture device and/or a display device.

In another aspect of the present disclosure, the computer-implemented method further includes one or more of: (1) in a case where the result of the comparing indicates that the observer is correctly positioned for perception of stereoscopic visual content, providing the message in the form of display content indicating that the observer is correctly positioned for perception of stereoscopic visual content; and/or (2) in a case where the result of the comparing indicates that the observer is correctly positioned for perception of stereoscopic visual content, providing the message in the form of audio content indicating that the observer is correctly positioned for perception of stereoscopic visual content.

In another aspect of the present disclosure, the predetermined position criterion includes a range of acceptable observer positions for perception of stereoscopic visual content or a range of unacceptable observer positions for perception of stereoscopic visual content.

According to another aspect of the present disclosure, a non-transitory computer-readable medium is provided that stores instructions which, when executed by a processor, cause an image capture device to capture an image of an observer, and cause the processor to determine a position of the observer based on the captured image, compare the determined position of the observer to a predetermined position criterion, and cause a message based on a result of the comparing to be provided to the observer.

In another aspect of the present disclosure, the instructions, when executed by the processor, further cause one or more of: (1) a display device to, in a case where the result of the comparing indicates that the position of the observer should be corrected for improved perception of stereoscopic visual content, provide the message in the form of display content indicating a direction in which the observer should move to correct position for improved perception of stereoscopic visual content; and/or (2) an audio device to, in a case where the result of the comparing indicates that the position of the observer should be corrected for improved perception of stereoscopic visual content, provide the message in the form of audio content indicating a direction in which the observer should move to correct position for improved perception of stereoscopic visual content.

In still another aspect of the present disclosure, the determining the position of the observer includes determining a relative position of one or more of an eye of the observer, stereoscopic eyeglasses worn by the observer, and/or a head of the observer, with respect to one or more of the image capture device and/or the display device.

In another aspect of the present disclosure, the instructions, when executed by the processor, further cause one or more of: (1) a display device to, in a case where the result of the comparing indicates that the observer is correctly positioned for perception of stereoscopic visual content, provide the message in the form of display content indicating that the observer is correctly positioned for perception of stereoscopic visual content; and/or (2) an audio device to, in a case where the result of the comparing indicates that the observer is correctly positioned for perception of stereoscopic visual content, provide the message in the form of audio content indicating that the observer is correctly positioned for perception of stereoscopic visual content.

In another aspect of the present disclosure, the predetermined position criterion includes a range of acceptable observer positions for perception of stereoscopic visual content or a range of unacceptable observer positions for perception of stereoscopic visual content.

According to another aspect of the present disclosure, a system for improving perception of stereoscopic visual content is provided that includes a display device, an image capture device configured to capture an image of an observer, and a processor configured to: determine a position of the observer based on the captured image of the observer, compare the determined position of the observer to a predetermined position criterion, and cause the display device to be repositioned based on a result of the comparing.

In another aspect of the present disclosure, the position of the observer includes a relative position of one or more of an eye of the observer, stereoscopic eyeglasses worn by the observer, and/or a head of the observer, relative to one or more of a position of the image capture device and/or a position of the display device.

In still another aspect of the present disclosure, the predetermined position criterion includes at least one acceptable observer position for perception of stereoscopic visual content, relative to one or more of a position of the image capture device and/or a position of the display device. The comparing the determined position of the observer to the predetermined position criterion includes computing a difference between the determined position of the observer and the acceptable observer position for perception of stereoscopic visual content.

In another aspect of the present disclosure, the causing the display device to be repositioned includes causing the display device to be repositioned to a position that decreases the difference between the determined position of the observer and the acceptable observer position for perception of stereoscopic visual content to within a predetermined threshold.

In another aspect of the present disclosure, the causing the display device to be repositioned includes causing the display device to be repositioned only if the computed error exceeds the predetermined threshold.

According to another aspect of the present disclosure, a computer-implemented method for improving perception of stereoscopic visual content is provided that includes capturing an image of an observer via an image capture device, determining a position of the observer based on the captured image of the observer, comparing the determined position of the observer to a predetermined position criterion, and causing a display device to be repositioned based on a result of the comparing.

In another aspect of the present disclosure, the position of the observer includes a relative position of one or more of an eye of the observer, stereoscopic eyeglasses worn by the observer, and/or a head of the observer, relative to one or more of a position of the image capture device and/or a position of the display device.

In still another aspect of the present disclosure, the predetermined position criterion includes at least one acceptable observer position for perception of stereoscopic visual content, relative to one or more of a position of the image capture device and/or a position of the display device. The comparing the determined position of the observer to the predetermined position criterion includes computing a difference between the determined position of the observer and the acceptable observer position for perception of stereoscopic visual content.

In another aspect of the present disclosure, the causing the display device to be repositioned includes causing the display device to be repositioned to a position that decreases the difference between the determined position of the observer and the acceptable observer position for perception of stereoscopic visual content to within a predetermined threshold.

In another aspect of the present disclosure, the causing the display device to be repositioned includes causing the display device to be repositioned only if the computed error exceeds the predetermined threshold.

According to another aspect of the present disclosure, a non-transitory computer-readable medium is provided that store instructions which, when executed by a processor, cause an image capture device to capture an image of an observer, and cause the processor to determine a position of the observer based on the captured image of the observer, compare the determined position of the observer to a predetermined position criterion, and cause a display device to be repositioned based on a result of the comparing.

In another aspect of the present disclosure, the position of the observer includes a relative position of one or more of an eye of the observer, stereoscopic eyeglasses worn by the observer, and/or a head of the observer, relative to one or more of a position of the image capture device and/or a position of the display device.

In still another aspect of the present disclosure, the predetermined position criterion includes at least one acceptable observer position for perception of stereoscopic visual content, relative to one or more of a position of the image capture device and/or a position of the display device. The comparing the determined position of the observer to the predetermined position criterion includes computing a difference between the determined position of the observer and the acceptable observer position for perception of stereoscopic visual content.

In another aspect of the present disclosure, the causing the display device to be repositioned includes causing the display device to be repositioned to a position that decreases the difference between the determined position of the observer and the acceptable observer position for perception of stereoscopic visual content to within a predetermined threshold.

In another aspect of the present disclosure, the causing the display device to be repositioned includes causing the display device to be repositioned only if the computed error exceeds the predetermined threshold.

### BRIEF DESCRIPTION OF THE DRAWINGS

The above and other aspects, features, and advantages of the present disclosure will become more apparent in light of the following detailed description when taken in conjunction with the accompanying drawings, in which:

FIG. 1 is a diagram of an exemplary robotic surgical system including a stereoscopic display in accordance with an embodiment of the present disclosure;

FIG. 2 illustrates additional aspects of an example stereoscopic display system shown in FIG. 1;

FIG. 3 illustrates additional aspects of the display device of the stereoscopic display system shown in FIG. 2;

FIG. 4 depicts a computer-implemented procedure for controlling the display device in accordance with a first example embodiment herein;

FIG. 5 depicts a computer-implemented procedure for controlling the display device in accordance with a second example embodiment herein; and

FIG. 6 depicts a computer-implemented procedure for controlling the display device in accordance with a third example embodiment herein.

### DETAILED DESCRIPTION

As used herein, the terms “clinician,” “surgeon,” “observer,” generally refer to a user of a stereoscopic display device described herein. Additionally, although the terms “first eye” and “second eye” are used herein to refer to a left eye and a right eye, respectively, of an observer, this use is provided by way of example and should not be construed as limiting.

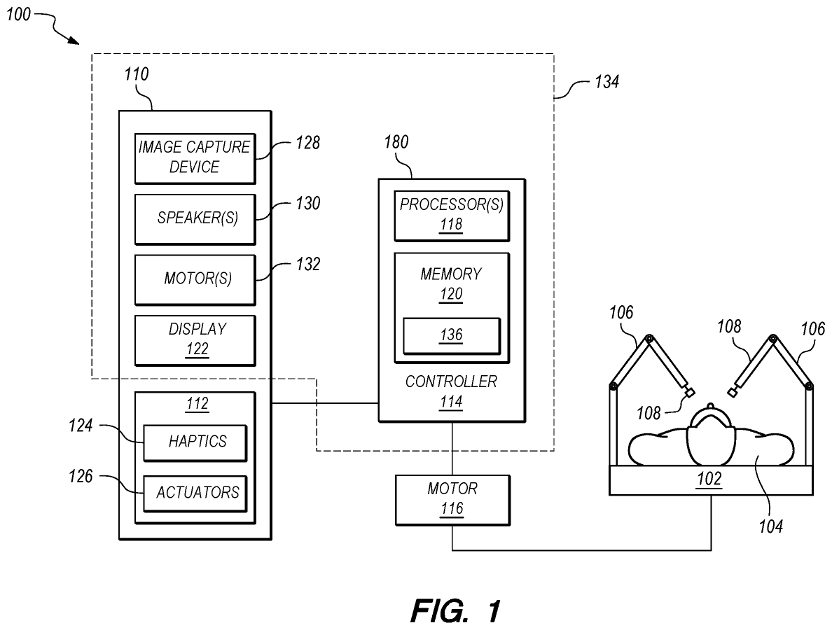

FIG. 1 shows an example robotic surgical system 100 that may be employed in accordance with various example embodiments herein. The specific number of components of the system 100 shown in FIG. 1 and the arrangement and configuration thereof are provided for illustrative purposes only, and should not be construed as limiting. For instance, various embodiments herein employ fewer or greater than all of the components shown in FIG. 1. Additionally, the example robotic surgical system 100 depicted in FIG. 1 is provided as an example context in which various example embodiments herein are applicable. However, the various example embodiments herein are also applicable in contexts other than robotic surgical systems, for instance, in general stereoscopic display contexts.

The system 100 includes an operating table 102 upon which a patient 104 lies during a surgical procedure, robotic arms 106 having corresponding surgical instruments 108 interchangeably fastened thereto, a console 110 having handles 112 with which a clinician (also referred to herein as an “observer”) interacts during the surgical procedure, and a controller 114 and one or more motors 116 by which the console 110 is coupled to the robotic arms 106 and surgical instruments 108. The robotic arms 106 are affixed to the operating table 102 and/or are arranged adjacent to the operating table 102 within range of the patient 104 undergoing the surgical procedure.

The controller 114 includes one or more processors 118 and memories 120, and may be integrated with the console 110 or provided as a standalone device within the operating theater. As described in further detail below, the processor 118 executes instructions 136 (in an example, software) stored in the memory 120 to perform procedures of the various embodiments herein. As will be appreciated, the processor 118 and memory 120 implementation is provided by way of example only, and should not be construed as limiting. For instance, procedures of any of the embodiments of the present disclosure may be implemented by hardware components, firmware components, software components, and/or any combination thereof.

During operation of the surgical system 100, the handles 112 are moved by the clinician to produce a corresponding movement and/or actuation of the working ends of the robotic arms 106 and/or surgical instruments 108. The handles 112 provide a signal to the controller 114 which then provides a corresponding signal to one or more drive motors 116. The one or more drive motors 116 are coupled to the robotic arms 106 in order to move the robotic arms 106 and/or surgical instruments 108.

The handles 112 may include various haptics 124 to provide feedback to the clinician relating to various tissue parameters or conditions, in an example, tissue resistance due to manipulation, cutting, or otherwise treating, pressure by the instrument onto the tissue, tissue temperature, tissue impedance, etc. As can be appreciated, such haptics 124 provide the clinician with enhanced tactile feedback simulating actual operating conditions. The haptics 124 may include vibratory motors, electroactive polymers, piezoelectric devices, electrostatic devices, subsonic audio wave surface actuation devices, reverse-electrovibration, or any other device capable of providing a tactile feedback to a user. The handles 112 may also include a variety of different actuators 126 for delicate tissue manipulation or treatment further enhancing the clinician's ability to mimic actual operating conditions.

The surgical instruments 108 may be any type of surgical instrument, such as, by way of example and not limitation, an image capture device, a probe, an end effector, a grasper, a knife, scissors, and/or the like. In accordance with some embodiments herein, one or more of the surgical instruments 108 may be a probe that includes a stereoscopic image capture device. The probe is inserted into a patient in order to capture a stereoscopic image of a region of interest inside the patient during a surgical procedure. In accordance with some embodiments herein, the stereoscopic images captured by the image capture device are communicated to a stereoscopic display device 122 (also referred to herein as a “display device” or simply a “display”) of the console 110 that displays the images to the clinician by way of stereoscopic eyeglasses (not shown in FIG. 1) worn by the clinician.

As described in further detail below, in some exemplary embodiments herein the console 110 includes an image capture device 128 (in an example, a camera) that captures an image of the observer (not shown in FIG. 1). The image capture device 128 can be integrated with, and/or positionally fixed to, the display 122, such that the positional relationship between the image capture device 128 and the display 122 is known and can be relied upon by the processor 118 in various computations. In one example, the processor 118 utilizes the image captured by the image capture device 128 to determine a position of the observer, compare the determined position of the observer to a predetermined position criterion, and cause a message based on a result of the comparing to be provided to the observer, for example, visibly by way of the display 122, audibly by way of one or more audio devices (in an example, speakers) 130, and/or through tactile feedback by way of the handles 112. Providing the observer with such a message can, if warranted, inform the observer how to move to a position that is more optimal for improved perception of stereoscopic visual content.

As also described in further detail below, in another exemplary embodiment herein the console 110 also includes one or more motors 132 configured to reposition the display 122, and the processor 118 is configured to determine a position of the observer based on the captured image of the observer, compare the determined position of the observer to a predetermined position criterion, and cause the motors 132 to reposition the display 122 based on a result of the comparing. The one or more motors 132 can be single-axis motors or multiple-axis (in an example, 3 axis) motors that facilitate repositioning of the display 122 along a single axis or along multiple axes, respectively. Repositioning the display 122 based on observer position can enable the display 122 to maintain a position, relative to the observer, that is more optimal for improved perception of stereoscopic visual content.

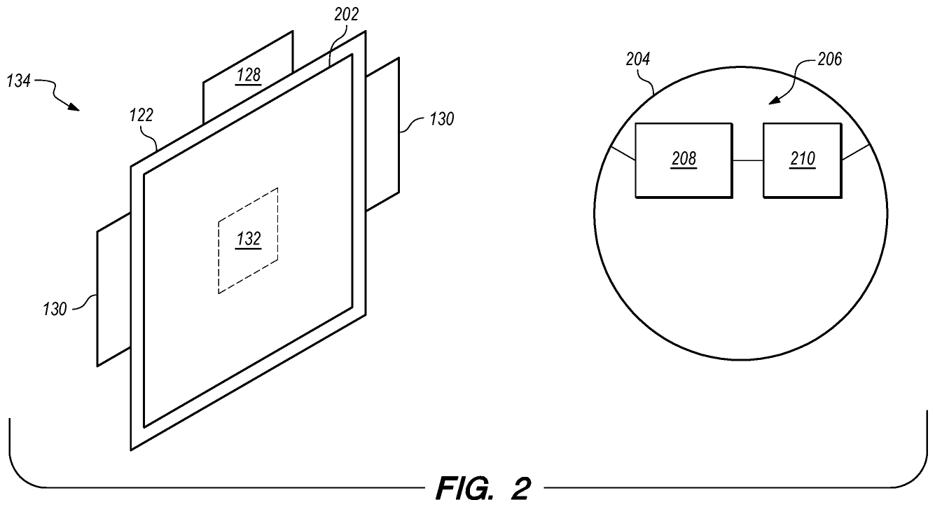

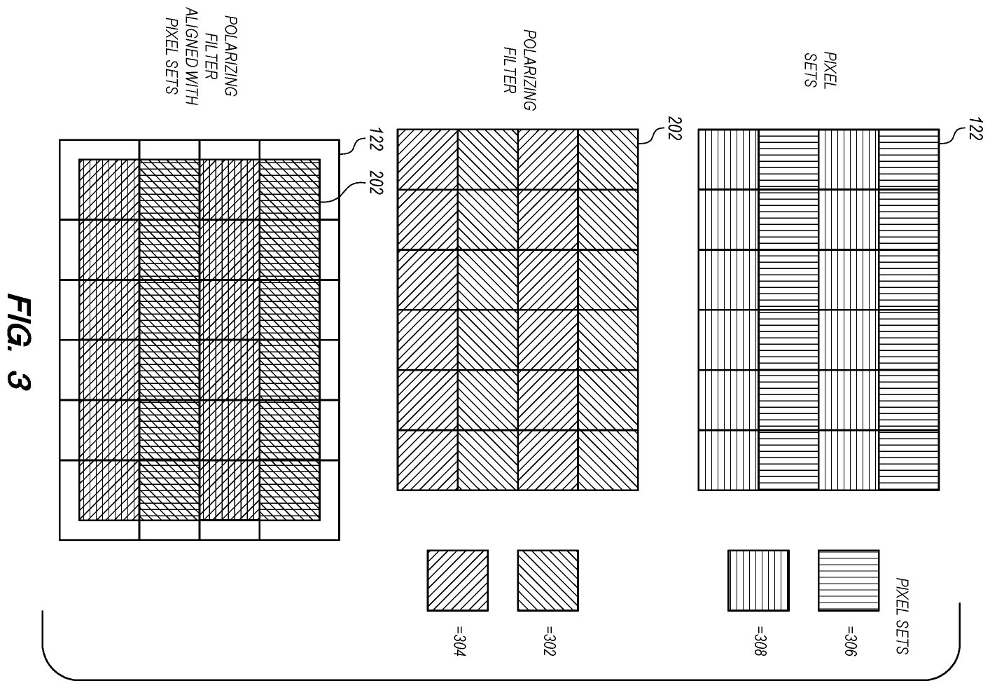

Certain components of the system 100 (for example, components 114, 118, 120, 122, 128, 130, and/or 132) may represent a stereoscopic display system 134 in accordance with some example embodiments herein. Reference will now be made to FIG. 2 and FIG. 3, which illustrate additional aspects of the example stereoscopic display system 134 and the display device 122 thereof. The specific number of components of the system 134 shown in FIG. 2 and FIG. 3, and the arrangement and configuration thereof, are provided for illustrative purposes only, and should not be construed as limiting. For instance, some embodiments herein employ fewer or greater components than the components shown in FIG. 2 and FIG. 3. Additionally, for clarity, some components of the system 134 are omitted from FIG. 2 and FIG. 3. Further, the stereoscopic display system 134 herein is also applicable in contexts other than robotic surgical systems, for instance, in general stereoscopic display contexts.

FIG. 2 includes a perspective view of a portion of the stereoscopic display system 134, showing an example arrangement of the image capture device 128, the audio devices 130, the motor(s) 132, and a polarizing filter 202, in accordance with various embodiments herein. Aspects of the polarizing filter 202, which may be affixed to, or integrated with, a screen of the display device 122, are shown in FIG. 3. The exemplary embodiment shown in FIG. 3 illustrates a portion of the display device 122, the portion including four rows of pixels, each row being six pixels wide. The polarizing filter 202 is aligned with the pixels of the display device 122 so as to direct visual content displayed by certain pixels to certain eyes of the observer 204 by way of the lenses 208 and 210 of the corresponding pair of stereoscopic eyeglasses 206 worn by the observer 204. In particular, as depicted in FIG. 3, the polarizing filter 202 includes a first portion 302 and a second portion 304. The first portion 302 of the polarizing filter 202 is aligned with a first set of pixels 306 (in an example, odd pixel rows) of the display device 122 and filters visual content displayed by the first set of pixels 306 according to a first polarization. The second portion 304 of the polarizing filter 202 is aligned with a second set of pixels 308 (in an example, even pixel rows) of the display device 122 and filters visual content displayed by the second set of pixels 308 according to a second polarization.

Referring back to FIG. 2, as briefly mentioned above, the stereoscopic eyeglasses 206 worn by the observer 204 of the display device 122 includes left and right lenses 208, 210. The left lens 208 of the eyeglasses 206 is matched to the first type of polarization and is designed to permit visual content polarized according to the first type of polarization (in an example, visual content displayed by the first set of pixels 306, or odd pixel rows) to reach the left eye and prevent visual content polarized according to the second type of polarization (in an example, visual content displayed by the second set of pixels 308, or even pixel rows) from reaching the left eye. The right lens 210 of the eyeglasses 206 is matched to the second type of polarization and is designed to permit the visual content polarized according to the second type of polarization to reach the right eye and prevent the visual content polarized according to the first type of polarization from reaching the right eye. The matching of the left lens 208 and right lens 210 of the eyeglasses to the first type of polarization and second type of polarization, respectively, is provided as a non-limiting example.

The first and second polarizations may be any mutually distinct types of polarizations. For instance, the first and second polarizations may be clockwise circular polarization and counterclockwise circular polarization, respectively. Alternatively, the first and second polarizations may be a first linear polarization and a second linear polarization, respectively, with the second linear polarization being ninety degrees out of phase with respect to the first linear polarization.

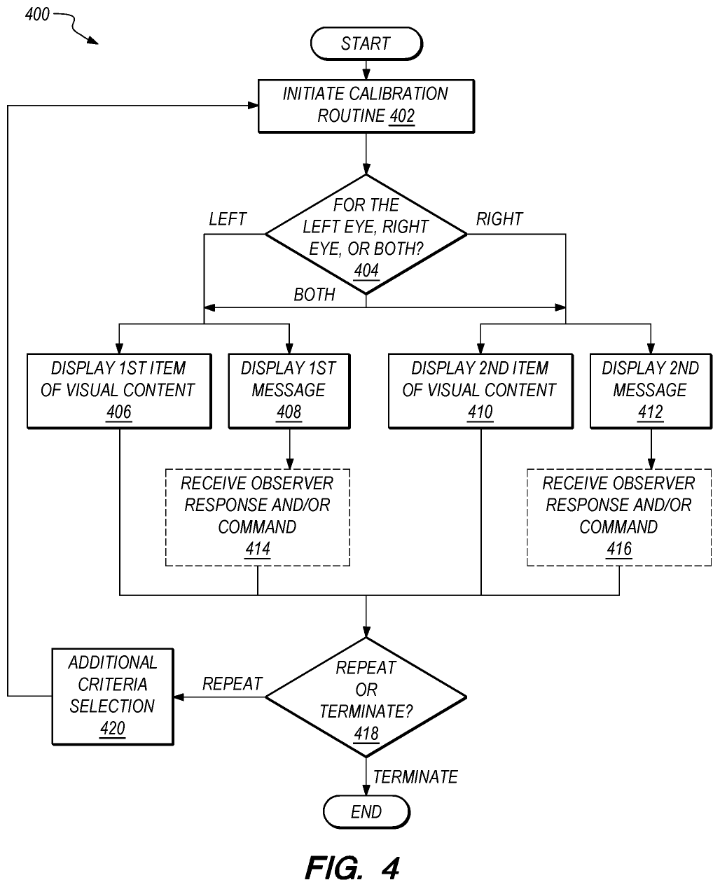

Having described an example stereoscopic display system 134, reference will now be made to FIG. 4, which depicts an example computer-implemented procedure 400 for controlling the stereoscopic display device 134, in accordance with a first embodiment herein. The procedure 400 may be implemented, at least in part, by the processor 118 executing instructions 136 stored in the memory 120 (FIG. 1). Additionally, the particular sequence of steps shown in the procedure 400 of FIG. 4 is provided by way of example and not limitation. Thus, the steps of the procedure 400 may be executed in sequences other than the sequence shown in FIG. 4 without departing from the scope of the present disclosure. Further, some steps shown in the procedure 400 of FIG. 4 may be concurrently executed with respect to one another instead of sequentially executed with respect to one another.

At block 402, a calibration routine for ensuring proper alignment of the observer 204 for improved perception of stereoscopic visual content is initiated, either automatically—for example, upon the display device 122 being powered on—or in response to a command—for example, a command inputted by the observer 204 by way of a user input device (in an example, a mouse, keyboard, touchscreen, microphone, and/or any other suitable user input device communicably coupled to the processor 118). Control then passes to block 404.

At block 404, a determination is made as to whether to execute the calibration routine for the left eye, the right eye, or both eyes simultaneously. The determination at block 404 may be made automatically or in response to a command. For example, the processor 118 may be configured to automatically implement the calibration routine multiple times in sequence, such as first implementing the calibration sequence for the left eye, then doing so for the right eye, and then doing so for both eyes simultaneously. As an alternative, the determination at block 404 may be made in response to a command, inputted by the observer 204 by way of a user input device, selecting one of the calibration routines.

If it is determined at block 404 that the calibration routine for the left eye is to be executed, then control passes to block 406 and block 408 to execute the calibration routine for the left eye.

At block 406, a first item of visual content (in an example, visual content intended to be visible by the left eye) is displayed through the first portion 302 of the polarizing filter 202, which is aligned with the first set of pixels 306 of the display device 122 and filters the first item of visual content according to the first polarization. The first item of visual content, for example, may include one or more of a predetermined color, a predetermined pattern, and/or predetermined textual content, that is readily perceptible by the observer 204.

At block 408, a first message is displayed through the first portion 302 of the polarizing filter 202 and/or through the second portion 304 of the polarizing filter 202. In this manner, the first message may reach one or both of the eyes of the observer 204 by way of one or both of the lenses 208 and 210, respectively, of the stereoscopic eyeglasses 206. In one example, the content of the first message is based on whether the first item of visual content is intended to be visible by the first eye of the observer 204 or the second eye of the observer. For example, the first message may indicate that the first item of visual content (in an example, an image of a triangle) is intended to be visible by the first eye (in an example, the left eye) of the observer 204, but not the second eye (in an example, the right eye) of the observer 204.

The perception by the observer 204 of the first item of visual content depends, at least in part, on the position and/or orientation of the eyes of the observer 204. For instance, if the eyes of the observer 204 are positioned in a proper position and orientation relative to the plane of the display 122 (for example, as described in further detail below), then the first item of visual content may be substantially completely visible by the first eye and substantially invisible by the second eye by way of the first and second lenses 208, 210 of the eyeglasses, respectively. However, when the eyes of the observer 204 are positioned in an improper position and/or orientation relative to the plane of the display 122, the perception by the observer 204 of the first item of visual content may be degraded through ghosting, where the eyeglasses 206 permit at least a portion of the first item of visual content to be at least partially visible by the second eye of the observer 204 and/or partially invisible by the first eye of the observer 204.

In one aspect, the first message includes a query and/or an instruction relating to repositioning of the eyes of the observer 204 so that the first item of visual content is visible by the first eye of the observer 204 but not visible by the second eye of the observer 204. For instance, the first message may instruct the observer 204 to alternate closing their left eye and their right eye and to reposition their head and/or eyes until the first item of visual content is visible by their left eye, by way of the first lens 208 of the eyeglasses 206, but is not visible by their right eye, by way of the second lens 210 of the eyeglasses 206. In another example, the first message may include a pair of images that appear as separate images when the eyes of the observer 204 are positioned in an improper observer position, and that converge (appear aligned and/or as a single combined image) when the eyes of the observer 204 are positioned in the proper position. In still another example, the first message may be actively controlled and/or varied based on a position of the observer 204 determined, for instance, by the image capture device 128. In this manner, the positional relationship between the eyes of the observer 204 and the stereoscopic display 122 may be optimized, thereby improving the perception by the observer 204 of stereoscopic visual content.

From block 408, control may pass from block 408 to optional block 414. At block 414, user input may be received from the observer 204 by way of a user input device (in an example, a mouse, keyboard, touchscreen, microphone, and/or any other suitable user input device communicably coupled to the processor 118). For instance, the user input may be a response to a query provided at block 408 as part of the first message regarding whether the first item of visual content is visible by the intended eye of the observer 204. The user input may alternatively or additionally include a command to cause an item of visual content to be displayed that is different (in an example, in color, in shape, in size, in which eye the content is intended to be visible by, or in another characteristic) from the first item of visual content. Control then passes to block 418, which is described in further detail below. Alternatively, in an embodiment, from block 408, control may pass directly to block 418 without execution of the routines of block 414.

Referring back to block 404, if it is determined that the calibration routine for the right eye is to be executed, then control passes to block 410 and block 412, to execute the calibration routine for the right eye.

At block 410, a second item of visual content (in an example, visual content intended to be visible by the right eye) is displayed through the second portion 304 of the polarizing filter 202, which is aligned with the second set of pixels 308 of the display device 122 and filters the second item of visual content according to the second polarization.

In general, the first item of visual content is distinct from the second item of visual content, to enable the observer 204 to distinguish between items of visual content intended to be visible by particular eyes of the observer 204. For instance, the first item of visual content and the second item of visual content, in some examples, may include respective predetermined colors, predetermined patterns, and/or predetermined textual content, that are readily distinguishable from one another by the observer.

At block 412, a second message is displayed through the first portion 302 of the polarizing filter 202 and/or through the second portion 304 of the polarizing filter 202. In this manner, the second message may reach one or both of the eyes of the observer 204 by way of one or both of the lenses 208 and 210 of the stereoscopic eyeglasses 206. In one example, the content of the second message is based on whether the second item of visual content is intended to be visible by the first eye of the observer 204 or the second eye of the observer. For example, the second message may indicate that the second item of visual content (in an example, an image of a circle) is intended to be visible by the second eye (in an example, the right eye) of the observer 204, but not the first eye (in an example, the left eye) of the observer 204.

As described above in the context of the first item of visual content, the perception by the observer 204 of the second item of visual content depends, at least in part, on the position and/or orientation of the eyes of the observer 204. In one aspect, the second message includes a query and/or an instruction relating to repositioning of the eyes of the observer 204 so that the second item of visual content is visible by the second eye of the observer 204 but not visible by the first eye of the observer 204. For instance, the second message may instruct the observer 204 to reposition their head and/or eyes until the second item of visual content is visible by their right eye, by way of the second lens 210 of the eyeglasses 206, but is not visible by their left eye, by way of the first lens 208 of the eyeglasses 206. In this manner, the positional relationship between the eyes of the observer 204 and the stereoscopic display 122 may be optimized, thereby improving the perception by the observer 204 of stereoscopic visual content.

From block 412, control may pass from block 412 to optional block 416. At block 416, user input may be received from the observer 204 by way of a user input device (in an example, a mouse, keyboard, touchscreen, microphone, and/or any other suitable user input device communicably coupled to the processor 118). For instance, the user input may be a response to a query provided at block 412 as part of the second message regarding whether the second item of visual content is visible by the intended eye of the observer 204. The user input may alternatively or additionally include a command to cause an item of visual content to be displayed that is different (in an example, in color, in shape, in size, in which eye the content is intended to be visible by, or in another characteristic) from the second item of visual content. Control then passes to block 418, which is described in further detail below. Alternatively, control from block 412 may pass directly to block 418, which is described below.

Referring back to block 404, if it is determined that the calibration routine is to be executed for both the left eye and the right eye simultaneously, then control passes from block 404 to block 406, block 408, block 410, and block 412, to execute (simultaneously and/or in succession) the calibration routine for both the left eye and the right eye, in the respective manners described above. In one example, the first item of visual content, the second item of visual content, the first message, and/or the second message are displayed concurrently with another one or more of the first item of visual content, the second item of visual content, the first message, and/or the second message. From block 406 and block 410, control passes to block 418. From block 408 and/or block 412, in an embodiment, control may pass to block 414 and/or block 416, respectively, to execute the respective routines described above. Alternatively, control from block 408 and/or block 412 may pass directly to block 418.

At block 418, the observer 204 is presented (in an example, visually via the display device 122, audibly via the speakers 130, and/or through tactile feedback by way of the handles 112) with an option to repeat the procedure 400 (in an example, if performed for one of the eyes, for another eye, or if performed with an item of visual content, with another item(s) of visual content) or to terminate the procedure 400. User input may be received from the observer 204, by way of a user input device (in an example, a mouse, keyboard, touchscreen, microphone, and/or any other suitable user input device communicably coupled to the processor 118), selecting whether to repeat the procedure 400 or terminate the procedure 400. If at block 418, user input is received opting to terminate the procedure 400, then the procedure terminates. If, on the other hand, at block 418 user input is received opting to repeat the procedure 400, then control passes to block 420.

At block 420, the observer 204 is presented (in an example, visually via the display device 122, audibly via the speakers 130, and/or through tactile feedback by way of the handles 112) with an option to select one or more criteria to be utilized during a subsequent iteration of the procedure 400. For instance, the observer 204 can select a particular eye to undergo the calibration routine, one or more particular item(s) of visual content, and/or other criteria to be utilized during the subsequent iteration of the procedure 400. Control then passes to block 402, as described above.

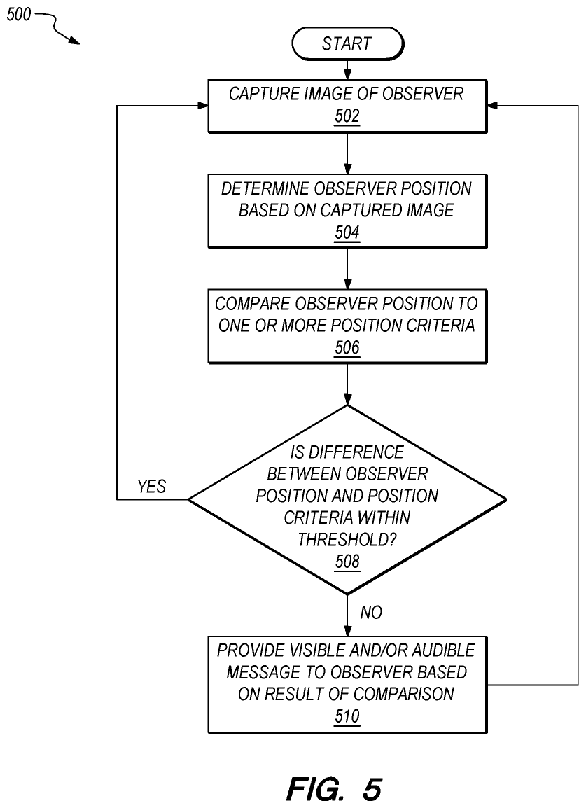

Having described an example computer-implemented procedure 400 for controlling the stereoscopic display device 134 in accordance with a first embodiment herein, reference will now be made to FIG. 5, which depicts an example computer-implemented procedure 500 for controlling the stereoscopic display device 134 in accordance with a second embodiment herein. The procedure 500 may be implemented, at least in part, by the processor 118 executing instructions 136 stored in the memory 120 (FIG. 1). Additionally, the particular sequence of steps shown in the procedure 500 of FIG. 5 is provided by way of example and not limitation. The steps of the procedure 500 may be executed in sequences other than the sequence shown in FIG. 5 without departing from the scope of the present disclosure. Further, some steps shown in the procedure 500 of FIG. 5 may be concurrently executed instead of sequentially executed.

At block 502, an image of at least a portion (in an example, a face) of the observer 204 is captured by the image capture device 128.

At block 504, a position and/or orientation of the observer 204 is determined based on the image that was captured at block 502. Although the terms “position,” “orientation,” and “pose” are used in various portions of the present disclosure, as a person having ordinary skill in the art would appreciate, in at least some portions of the present disclosure the terms “position” and “orientation” and/or the term “pose,” which generally refers to both position and orientation, may be used interchangeably. The determining of the position of the observer 204 performed at block 504 may include, for instance, determining a relative position of one or more of (1) the eyes of the observer 204, (2) the stereoscopic eyeglasses 206 worn by the observer 204, and/or (3) a head of the observer 204, and/or (4) another feature, such as a nose, of the observer 204, with respect to the image capture device 128 and/or the display device 122. For example, one or more known tracking algorithms (in an example, head tracking, eye tracking, eyeglasses tracking, and/or the like) are employed to determine the position of the observer 204 at block 504. For instance, the tracking algorithm may be based on rigid body tracking as estimated from three or more markers, which may be included on eyeglasses, or may be based on geometric feature extraction, such as eye and/or nose extraction, which is used to estimate head position or pose. In one example, the positional relationship between the image capture device 128 and the display device 122, whether fixed or variable, is known and is utilized at block 504 to determine the position of the observer 204 relative to the display device 122. For instance, the positional relationship between the image capture device 128 and the display device 122 can be assumed to within some tolerance based on the design and manufacture specifications of the display device 122. Alternatively or additionally, the positional relationship between the image capture device 128 and the display device 122 can be measured by, for example, positioning a set of shapes in a known position relative to the image capture device 128 and/or to the display device 122 (for instance, by attaching to the image capture device 128 and/or to the display device 122 a calibration jig that includes the set of shapes), utilizing the image capture device 128 to capture images of the shapes, and utilizing the processor 118 to determine, based on the captured images of the shapes, the position and/or orientation of the front surface of the display device 122 in relation to the image capture device 128.

At block 506, the position and/or orientation of the observer 204 that was determined at block 504 is compared to one or more predetermined position criteria, orientation criteria, and/or pose criteria. The predetermined position criteria, in some examples, includes a range of acceptable observer positions (which may also be referred to as a proper observer position) for perception of stereoscopic visual content and/or a range of unacceptable observer positions (which may also be referred to as an improper observer position) for perception of stereoscopic visual content. For example, the range of acceptable observer positions may be defined to include an ideal observer position and a range of positions that deviate from the ideal position in one, two, and/or three dimensions (for example, in an x-direction, y-direction, and/or z-direction of a coordinate system relative to the display device 122) by one or more respective predetermined allowable amounts or tolerance amounts; and the range of unacceptable observer positions may be defined to include all positions that are not included in the range of acceptable observer positions. In one example, the ideal observer position may be defined as a position where the observer is facing the display device 122, is vertically and horizontally centered with respect to the display device 122, and is positioned such that a plane defined by the front center surfaces of the eyes of the observer is parallel to a plane defined by the front surface of the display device 122 and is distanced from the plane defined by the front surface of the display device 122 by a predetermined recommended viewing distance, and such that a line defined by the front center surfaces of the eyes of the observer is parallel to a line defined by a horizontal edge of the display device 122. In some examples, different tolerance amounts are employed for different dimensions or directions. For instance, in cases where a particular amount of horizontal deviation from the ideal observer position is not as detrimental to optimal perception of stereoscopic content than a similar amount of vertical deviation would be, a tolerance amount employed for horizontal deviations from the ideal observer position may be larger than a tolerance amount employed for vertical deviations from the ideal observer position. The range of acceptable observer positions and/or the range of unacceptable observer positions, in some examples, is defined at least in part based on objectively measurable and/or numerical criteria. For instance, a range of acceptable observer positions may be defined based upon a recommended viewing range of 140 centimeters from the display device 122, and a vertical tolerance amount of plus or minus 10 degrees, in addition to other tolerance amounts for other respective dimensions or directions for example.

At block 508, depending on the result of the comparing performed at block 506, control is passed to either block 510 or back to block 502. For example, if the result of the comparing performed at block 506 indicates that the position of the observer 204 that was determined at block 504 is within a range of positions acceptable for proper perception of stereoscopic visual content, then control is passed back to block 502 to continually track the position of the observer 204 and provide the observer 204 with visual, audible, tactile, and/or any other type of feedback to enable the observer 204 to maintain proper position for perception of stereoscopic visual content provided by the display 122. In an embodiment, the result of block 506 indicates the position of the observer 204 is within a range of positions acceptable for proper perception of stereoscopic visual content when a difference between the determined position of the observer 204 and the range of acceptable positions falls within a predetermined threshold.

Although not shown in FIG. 5, in another example, the procedure 500 may include one or more additional operations. For example, the one or more additional operations may be added after block 508 and before control is passed back to block 502. In an embodiment, in a case where the result of the comparing performed at block 506 indicates that the observer 204 is correctly positioned for perception of stereoscopic visual content, a message is provided visually, by way of the display device 122, in the form of display content, audibly, by way of the speakers 130, and/or through tactile feedback by way of the handles 112, indicating that the observer 204 is correctly positioned for perception of stereoscopic visual content.

If, on the other hand, the result of the comparing performed at block 506 indicates that the position of the observer 204 that was determined at block 504 is outside of a range of positions acceptable for proper perception of stereoscopic visual content, then control is passed to block 510.

At block 510, a message based on the result of the comparing performed at block 506 is provided to the observer 204 (in an example, visually by way of the display 122, audibly by way of the speakers 130, and/or through tactile feedback by way of the handles 112). The message may indicate, for example, one or more corrective actions that the observer 204 should take to improve perception of stereoscopic visual content displayed by the display device 122. For instance, in a case where the result of the comparing performed at block 506 indicates that the position of the observer 204 should be corrected for improved perception of stereoscopic visual content, the message is provided, by way of the display device 122, in the form of display content indicating a direction in which the observer 204 should move to correct their position for improved perception of stereoscopic visual content. Alternatively or additionally, in a case where the result of the comparing performed at block 506 indicates that the position of the observer 204 should be corrected for improved perception of stereoscopic visual content, the message is audibly provided, by way of the speakers 130, in the form of audio content indicating a direction in which the observer 204 should move to correct their position for improved perception of stereoscopic visual content. Control then passes back to block 502, described above.

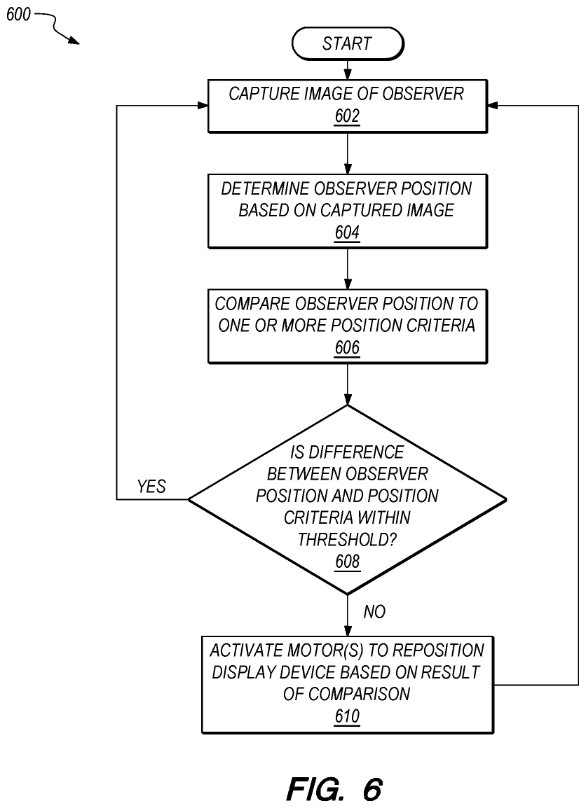

Having described example computer-implemented procedures 400 and 500 for controlling the stereoscopic display device 134 in accordance with first and second embodiments, respectively, reference will now be made to FIG. 6, which depicts an example computer-implemented procedure 600 for controlling the stereoscopic display device 134 in accordance with a third embodiment herein. The procedure 600 may be implemented, at least in part, by the processor 118 executing instructions 136 stored in the memory 120 (FIG. 1). Additionally, the particular sequence of steps shown in the procedure 600 of FIG. 6 is provided by way of example and not limitation. The steps of the procedure 600 may be executed in sequences other than the sequence shown in FIG. 6 without departing from the scope of the present disclosure. Further, some steps shown in the procedure 600 of FIG. 6 may be concurrently executed instead of sequentially executed.

At block 602, an image of at least a portion (in an example, a face) of the observer 204 is captured by the image capture device 128. For instance, the image of a head, an eye, and/or the eyeglasses 206 of the observer can be included in the captured image to enable tracking of the head, eye(s), and/or eyeglasses of the observer 204, as described below.

At block 604, a position of the observer 204 is determined based on the image that was captured at block 602. The determining of the position of the observer 204 performed at block 604 may include, for instance, determining a relative position of one or more of an eye of the observer 204, the stereoscopic eyeglasses 206 worn by the observer 204, and/or a head of the observer 204, with respect to the image capture device 128 and/or the display device 122. For example, as described in further detail above, one or more known tracking algorithms (in an example, head tracking, eye tracking, eyeglasses tracking, and/or the like) are employed to determine the position of the observer 204 at block 504. In one example, as also described in further detail above, the positional relationship between the image capture device 128 and the display device 122, whether fixed or variable, is known and is utilized at block 504 to determine the position of the observer 204 relative to the display device 122.

At block 606, the position of the observer 204 that was determined at block 604 is compared to one or more predetermined position criteria. As described in further detail above, the predetermined position criteria may include a range of acceptable observer positions for perception of stereoscopic visual content or a range of unacceptable observer positions for perception of stereoscopic visual content.

In one example, the predetermined position criterion includes at least one acceptable observer position for proper perception of stereoscopic visual content, relative to a position of the image capture device 128, and/or a position of the display device 122, and the comparing performed at block 606 includes computing a difference between the position of the observer 204 determined at block 604 and the at least one acceptable observer position.

At block 608, depending on the result of the comparing performed at block 606, control is passed to either block 610 or back to block 602. For example, if the result of the comparing performed at block 606 indicates that the position of the observer 204 that was determined at block 604 is within a range of positions acceptable for proper perception of stereoscopic visual content, then control is passed back to block 602 to continually cause the display device 122 to track the position of the observer 204 to maintain proper position for optimal perception of stereoscopic visual content provided by the display 122.

If, on the other hand, the result of the comparing performed at block 606 indicates that the position of the observer 204 that was determined at block 604 is outside of a range of positions acceptable for proper perception of stereoscopic visual content, then control is passed to block 610.

At block 610, the motors 132 are actuated to cause the display device 122 to be repositioned based on a result of the comparing that was performed at block 606. For example, the motors 132 may cause the display device 122 to be repositioned to a position that decreases the difference between the position of the observer determined at block 604 and the observer position deemed acceptable for proper perception of stereoscopic visual content to within a predetermined threshold (based on the predetermined position criterion utilized at block 606). In another example, hysteresis may be provided, whereby the display device 122 is repositioned only if the computed difference exceeds the predetermined threshold. In this manner, the position of the observer 204 can be continually tracked and the display device 122 can continually follow the tracked position of the observer 204 so as to maintain a proper positional relationship between the observer 204 and the display device 122 for optimal perception of stereoscopic visual content provided by the display device 122. Control then passes back to block 602, described above.

The embodiments disclosed herein are examples of the disclosure and may be embodied in various forms. For instance, although certain embodiments herein are described as separate embodiments, each of the embodiments herein may be combined with one or more of the other embodiments herein. Specific structural and functional details disclosed herein are not to be interpreted as limiting, but as a basis for the claims and as a representative basis for teaching one skilled in the art to variously employ the present disclosure in virtually any appropriately detailed structure. Like reference numerals may refer to similar or identical elements throughout the description of the figures.

The phrases “in an embodiment,” “in embodiments,” “in some embodiments,” or “in other embodiments” may each refer to one or more of the same or different embodiments in accordance with the present disclosure. A phrase in the form “A or B” means “(A), (B), or (A and B).” A phrase in the form “at least one of A, B, or C” means “(A); (B); (C); (A and B); (A and C); (B and C); or (A, B, and C).” The term “clinician” may refer to a clinician or any medical professional, such as a doctor, nurse, technician, medical assistant, or the like, performing a medical procedure.

The systems described herein may also utilize one or more controllers to receive various information and transform the received information to generate an output. The controller may include any type of computing device, computational circuit, or any type of processor or processing circuit capable of executing a series of instructions that are stored in a memory. The controller may include multiple processors and/or multicore central processing units (CPUs) and may include any type of processor, such as a microprocessor, digital signal processor, microcontroller, programmable logic device (PLD), field programmable gate array (FPGA), or the like. The controller may also include a memory to store data and/or instructions that, when executed by the one or more processors, causes the one or more processors to perform one or more methods and/or algorithms.

Any of the herein described methods, programs, algorithms or codes may be converted to, or expressed in, a programming language or computer program. The terms “programming language” and “computer program,” as used herein, each include any language used to specify instructions to a computer, and include (but is not limited to) the following languages and their derivatives: Assembler, Basic, Batch files, BCPL, C, C+, C++, Delphi, Fortran, Java, JavaScript, machine code, operating system command languages, Pascal, Perl, PL1, scripting languages, Visual Basic, metalanguages which themselves specify programs, and all first, second, third, fourth, fifth, or further generation computer languages. Also included are database and other data schemas, and any other meta-languages. No distinction is made between languages which are interpreted, compiled, or use both compiled and interpreted approaches. No distinction is made between compiled and source versions of a program. Thus, reference to a program, where the programming language could exist in more than one state (such as source, compiled, object, or linked) is a reference to any and all such states. Reference to a program may encompass the actual instructions and/or the intent of those instructions.

Any of the herein described methods, programs, algorithms or codes may be contained on one or more machine-readable media or memory. The term “memory” may include a mechanism that provides (in an example, stores and/or transmits) information in a form readable by a machine such a processor, computer, or a digital processing device. For example, a memory may include a read only memory (ROM), random access memory (RAM), magnetic disk storage media, optical storage media, flash memory devices, or any other volatile or non-volatile memory storage device. Code or instructions contained thereon can be represented by carrier wave signals, infrared signals, digital signals, and by other like signals.

It should be understood that the foregoing description is only illustrative of the present disclosure. Various alternatives and modifications can be devised by those skilled in the art without departing from the disclosure. Accordingly, the present disclosure is intended to embrace all such alternatives, modifications and variances. The embodiments described with reference to the attached drawing figures are presented only to demonstrate certain examples of the disclosure. Other elements, steps, methods, and techniques that are insubstantially different from those described above and/or in the appended claims are also intended to be within the scope of the disclosure.

1. A stereoscopic display system, comprising:
a display device including a first plurality of pixels and a second plurality of pixels;
a polarizing filter affixed to, or integrated with, the display device, the polarizing filter including:
a first portion that filters visual content according to a first polarization and is aligned with the first plurality of pixels of the display device, and
a second portion that filters visual content according to a second polarization and is aligned with the second plurality of pixels of the display device;
a memory storing instructions; and
a processor configured to execute the instructions to cause the display device to:
display a first item of visual content through the first portion of the polarizing filter, and
display, through at least one of the first portion of the polarizing filter or the second portion of the polarizing filter, a first message based on whether the first item of visual content is intended to be visible by a first eye of an observer or a second eye of the observer.

2. The system of claim 1, wherein the processor is further configured to execute the instructions to cause the display device to:
display a second item of visual content through the second portion of the polarizing filter, and
display, through at least one of the first portion of the polarizing filter or the second portion of the polarizing filter, a second message based on whether the second item of visual content is intended to be visible by the first eye of the observer or the second eye of the observer.

3. The system of claim 2, wherein at least one of the first item of visual content, the second item of visual content, the first message, or the second message are concurrently displayed with at least another one of the first item of visual content, the second item of visual content, the first message, or the second message.

4. The system of claim 1, wherein at least one of the first item of visual content or the second item of visual content includes at least one of a predetermined color, a predetermined pattern, or predetermined textual content, the first item of visual content being distinct from the second item of visual content.

5. The system of claim 1, wherein the first message includes at least one of a query or an instruction relating to repositioning of the eyes of the observer so that the first item of visual content is visible by the first eye of the observer but not visible by the second eye of the observer, and the second message includes at least one of a query or an instruction relating to repositioning of the eyes of the observer so that the second item of visual content is visible by the second eye of the observer but not visible by the first eye of the observer.

6. The system of claim 1, wherein the first polarization is clockwise circular polarization and the second polarization is counterclockwise circular polarization, or the first polarization is a first linear polarization and the second polarization is a second linear polarization ninety degrees out of phase with respect to the first linear polarization.

7. A computer-implemented method for controlling a stereoscopic display device, comprising:
displaying a first item of visual content through a first portion of a polarizing filter that filters visual content according to a first polarization and is aligned with a first plurality of pixels of a display device, and
displaying, through at least one of the first portion of the polarizing filter or a second portion of the polarizing filter that filters visual content according to a second polarization and is aligned with a second plurality of pixels of the display device, a first message based on whether the first item of visual content is intended to be visible by a first eye of an observer or a second eye of the observer.

8. The computer-implemented method of claim 7, further comprising:
displaying a second item of visual content through the second portion of the polarizing filter; and
displaying, through at least one of the first portion of the polarizing filter or the second portion of the polarizing filter, a second message based on whether the second item of visual content is intended to be visible by the first eye of the observer or the second eye of the observer.

9. The computer-implemented method of claim 8, wherein at least one of the first item of visual content, the second item of visual content, the first message, or the second message are concurrently displayed with at least another one of the first item of visual content, the second item of visual content, the first message, or the second message.

10. The computer-implemented method of claim 7, wherein at least one of the first item of visual content or the second item of visual content includes at least one of a predetermined color, a predetermined pattern, or predetermined textual content, the first item of visual content being distinct from the second item of visual content.

11. The computer-implemented method of claim 7, wherein the first message includes at least one of a query or an instruction relating to repositioning of the eyes of the observer so that the first item of visual content is visible by the first eye of the observer but not visible by the second eye of the observer, and the second message includes at least one of a query or an instruction relating to repositioning of the eyes of the observer so that the second item of visual content is visible by the second eye of the observer but not visible by the first eye of the observer.

12. The computer-implemented method of claim 7, wherein the first polarization is clockwise circular polarization and the second polarization is counterclockwise circular polarization, or the first polarization is a first linear polarization and the second polarization is a second linear polarization ninety degrees out of phase with respect to the first linear polarization.

13. A non-transitory computer-readable medium having stored thereon instructions which, when executed by a processor, cause a display device to:
display a first item of visual content through a first portion of a polarizing filter that filters visual content according to a first polarization and is aligned with a first plurality of pixels of a display device, and
display, through at least one of the first portion of the polarizing filter or a second portion of the polarizing filter that filters visual content according to a second polarization and is aligned with a second plurality of pixels of the display device, a first message based on whether the first item of visual content is intended to be visible by a first eye of an observer or a second eye of the observer.

14. The non-transitory computer-readable medium of claim 13, wherein the instructions, when executed by the processor, further cause the display device to:
display a second item of visual content through the second portion of the polarizing filter; and
display, through at least one of the first portion of the polarizing filter or the second portion of the polarizing filter, a second message based on whether the second item of visual content is intended to be visible by the first eye of the observer or the second eye of the observer.

15. The non-transitory computer-readable medium of claim 14, wherein at least one of the first item of visual content, the second item of visual content, the first message, or the second message are concurrently displayed with at least another one of the first item of visual content, the second item of visual content, the first message, or the second message.

16. The non-transitory computer-readable medium of claim 13, wherein at least one of the first item of visual content or the second item of visual content includes at least one of a predetermined color, a predetermined pattern, or predetermined textual content, the first item of visual content being distinct from the second item of visual content.

17. The non-transitory computer-readable medium of claim 13, wherein the first message includes at least one of a query or an instruction relating to repositioning of the eyes of the observer so that the first item of visual content is visible by the first eye of the observer but not visible by the second eye of the observer, and the second message includes at least one of a query or an instruction relating to repositioning of the eyes of the observer so that the second item of visual content is visible by the second eye of the observer but not visible by the first eye of the observer.

18. The non-transitory computer-readable medium of claim 13, wherein the first polarization is clockwise circular polarization and the second polarization is counterclockwise circular polarization, or the first polarization is a first linear polarization and the second polarization is a second linear polarization ninety degrees out of phase with respect to the first linear polarization.

19-48. (Canceled)
